(19) United States
(12) Patent Application Publication
(10) Pub. No.: US 2010/0324439 A1
(43) Pub. Date: Dec. 23, 2010

(54) HIGH FREQUENCY AIRWAY OSCILLATION FOR EXHALED AIR DIAGNOSTICS

(76) Inventor: Paul Wesley Davenport, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/797,000

(22) Filed: Jun. 9, 2010

Related U.S. Application Data
(60) Provisional application No. 61/218,750, filed on Jun. 19, 2009.

(51) Int. Cl. A61B 5/08

(52) U.S. Cl. 600/532

(57) ABSTRACT

The present invention relates to noninvasive methods for obtaining exhaled breath condensate (EBC) samples from the airways and lungs of a subject for use in diagnosing various conditions and diseases associated with biomarkers present in EBC, including diagnosis of lung cancer. The methods of the subject invention include generating and/or applying an oscillating airflow to a subject during inhalation and/or exhalation to induce increased concentration of biomarkers in EBC; collecting EBC from exhaled breath; and analyzing the collected EBC for biomarkers associated with disorders and/or diseases.

### CROSS-REFERENCE TO A RELATED APPLICATION

This application claims the benefit of U.S. provisional application Ser. No. 61/218,750, filed Jun. 19, 2009, which is incorporated herein by reference in its entirety.

### BACKGROUND OF INVENTION

There has been a recent increase in interest in providing noninvasive means for diagnosing various diseases and conditions in individuals. One method that has generated a great deal of interest involves induction and measurement of biomarkers in exhaled breath. By identifying and measuring the various biomarkers from the cooled and condensed exhaled breath, various conditions and diseases associated with the biomarkers can be non-invasively diagnosed. Unfortunately, very few pathology indicator molecules, cells and bacteria are in the lung air so a new method is needed to increase exhaled concentrations of cells, bacteria and substances lining the airways in the lung.

U.S. Pat. Nos. 6,379,316 and 7,018,348 describe noninvasive assays involving induced sputum discharge and analysis of sputum samples for markers of pulmonary disorders, especially lung cancer, present in the lower respiratory tract. Unfortunately, sputum samples often contain gross salivary and/or airway/lung material contaminants and are not the most reliable method for sampling the lining fluid of the lower respiratory tract. It is understood by the skilled artisan that exhaled breath condensates (EBC) collected through the mouth contain molecules not present in saliva and the electrolyte ratios of saliva differ from those in the orally collected condensate. This suggests that saliva is not the dominant contributor to EBC.

Unfortunately, progress in breath testing for various diseases and drug monitoring is hindered by the technical difficulty of obtaining sufficient concentrations of exhaled biomarkers in the breath (without assistance, nanomolar or picomolar concentrations of biomarkers are normally obtained). Research has been reported using breath sampling using large heated tubes (U.S. Pat. No. 5,465,728) and cylindrical (U.S. Pat. Nos. 6,244,096; 6,319,724; and 6,467,333) containers to collect desired portions of the breath for sampling. Unfortunately, these systems do not address the underlying problem that the resulting samples of EBC do not contain sufficient concentration of biomarkers of interest for measurement. There are no currently available systems for inducing and collecting a greater concentration of biomarkers in exhaled breath. What is desired is an optimized non-invasive sample collection system that induces exhalation of greater concentrations of biomarkers in breath for detection and measurement. In addition, it would be beneficial if the sample collection system is small in size, ideally hand-held or portable, without compromising sensitivity and selectivity of the biomarker of interest for detection.

### BRIEF SUMMARY

Embodiments of the invention are directed to systems and methods for increasing the concentration of biomarkers released in exhaled breath and collecting the same by generating and/or maintaining an air oscillatory component to a subject; collecting a sample of exhaled breath; and collecting and assessing exhaled breath condensates from the exhaled breath.

### BRIEF DESCRIPTION OF DRAWINGS

FIG. 1 shows an embodiment of a high frequency airway oscillation system for exhaled air diagnostics in accordance with the invention.

FIG. 2 shows another embodiment of a high frequency airway oscillation system for exhaled air diagnostics in accordance with the invention.

FIG. 3 shows yet another embodiment of a high frequency airway oscillation system for exhaled air diagnostics in accordance with the invention.

### DETAILED DISCLOSURE

The present invention has surprisingly found that the concentration of exhaled breath condensates (EBC) in oral exhaled breath is greatly increased by the presence of an oscillating airflow provided to subjects. Moreover, the invention increases the amount of substances exhaled that are normally present on the lining of the airways in the lung (such as cells and bacteria) and not normally exhaled in readily detectable concentrations. Thus, the subject invention increases the sensitivity of measurement of lung substances, cells, and organisms associated with infection.

According to the subject invention, methods for increasing the concentration of EBC in oral exhaled breath comprises the steps of: supplying an external means for generating and/or maintaining an oscillating airflow to a subject; collecting at least one exhaled breath sample following application of the oscillating airflow; and assessing the EBCs present in the exhaled breath sample. The EBC is analyzed for biomarkers, which can include identification and/or measurement of concentration of specific biomarkers present in the EBC. In yet another related embodiment, following EBC sample analysis, the subject is diagnosed with regard to health status, including diagnosis of any diseases and/or conditions associated with biomarkers present in the EBC.

The systems of the present invention for increasing the concentration of EBC in exhaled breath samples include the following parts: 1) an oscillating pressure means to be applied to the subject's airflow during inhalation and/or exhalation; and 2) an EBC collection means. Certain embodiments further comprise a sensor having the ability to detect and/or quantify biomarkers present in the condensates. In related embodiments, the sensor is coupled to a processor, which can store, track, trend, and interpret the sensor signals to provide useful information regarding biomarker amount or concentration for display to the user.

As defined herein, a subject is a mammal to which the means for maintaining and generating oscillating airflow is applied. Mammalian species that benefit from the disclosed systems and methods include, but are not limited to, humans, apes, chimpanzees, orangutans, monkeys, and domesticated animals (such as pets) such as dogs, cats, mice, rats, guinea pigs, hamsters, horses, cows, and anesthetized wild animals, including aquatic mammals.

According to the subject invention, exhaled breath comprises gaseous materials, such as carbon dioxide, NO, and oxygen, and non-gaseous materials, such as liquid droplets, insoluble and soluble substances, and other substances that are not volatile but may be suspended in exhaled breath at normal physiological (body) temperatures. Materials in the exhaled breath that are not in the gaseous state at the opening of the mouth or nose when exhaled are considered to be exhaled breath condensates (EBC) for the purposes of this invention. With exposure to high frequency airway oscillation, non-gaseous substances such as cells and bacteria that are attached to the walls of the airway are forced into the air of the airways and expelled out of the lung for sampling, identification, and/or clinical diagnosis. For example, EBC comprises, either alone or in combination, cells (such as alveolar macrophages, lung eosinophils, and bacteria), water vapor, airway droplets, solutes, nonvolatile molecules (which can be both hydrophobic and water-soluble molecules), as well as water-soluble volatile compounds that are absorbed by condensing water during collection of exhaled breath and/or EBC. In addition, the subject invention encompasses collection and/or detection of substances delivered to the lung by blood that are exhaled and detectable in EBC, including alcohol, intravenous anesthetics, and the like.

Specific biomarkers that are collected and measured in EBC for use in diagnosis of disease or condition in accordance with the subject invention include, but are not limited to, alveolar macrophages, lung eosinophils, bacteria, H₂O₂, adenosine, nitrate (NO₃⁻) and nitrite (NO₂⁻.), nitrotyrosine, nitrosothiols (RS-NOs), arachidonic acid metabolites (such as prostaglandins and thromboxanes), leukotrienes (such as leukotriene (LT)C₄, LTD₄, LT₄)), 8-isoprostanes, aldehydes (such as malondialdehyde, 4-hydroxyhexanal, 4-hydroxynonenal, hexanal, heptanal, and nonanal), ammonia (NH₃ and NH₄), cytokines, p53 mutation, DNA hepatocyte growth factor, vitronectin, endothelin1, chemotactic activity, DNA fragments, and proteins.

Diseases and conditions that can be diagnosed in accordance with the subject invention include, but are not limited to, inflammatory conditions, airway infections, common-cold, tumors, drug-related effects, and anatomical abnormalities. Specific diseases or conditions include, but are not limited to; asthma, cystic fibrosis, tuberculosis, chronic obstructive pulmonary diseases, bronchiectasis and acute respiratory distress syndrome, acute hypoxaemic respiratory failure, reperfusion injury, allergic rhinitis, system sclerosis, respiratory tract infection, bacterial pneumonia, interstitial lung disease, pulmonary sarcoidosis, obstructive sleep apnea, ozone-inhalation, acute lung injury, and respiratory cancers including lung cancer. All of these diseases or conditions can be diagnosed by analyzing EBC samples collected in accordance with the subject invention using morphologic, immunochemical, fluorescence, molecular, or genetic techniques.

According to the subject invention, the means for generating and/or maintaining an oscillating airflow are those that induce intra-thoracic oscillations. Intra-thoracic oscillations are generated orally, nasally and/or endotracheally and created using variable frequency and amplitude pressure or airflow pump producing air force waves within the airways generating controlled oscillating positive pressure. When oscillation frequency approximates the resonance frequency of the pulmonary system, endobronchial pressure oscillations are amplified and result in vibrations of the airways and lungs. The intermittent increases in endobronchial pressure reduce the collapsibility of the airways during exhalation, thereby mobilizing the release of increased concentration of EBC in exhaled breath as compared with EBC released in exhaled breath sampled without oscillating airflow. According to the subject invention, external means for generating and/or maintaining oscillating airflow does not include humming. Humming is normally used as a way to vibrate air and increase movement of molecules out of the nasal airways. In contrast, the external oscillating airflow means of the invention is a mechanical device that applies oscillating air force waves beyond the nasal passages and into the lungs and airways.

Methods and devices currently available for inducing intra-thoracic oscillations and for generating and/or maintaining oscillating airflow to a subject include, but are not limited to: flutter devices (devices that contain ball bearings that repeatedly interrupt the outward flow of air from a subject); acapella devices (flow operated oscillatory positive expiratory pressure (PEP) device that uses a counterweighted plug and magnet to generate oscillatory forces); cornet devices (tubes that house inner tubes where the rotation of the inner tube reflects resistance generated in airflow—as the subject exhales through the outer tube, the inner tube unfurls generating a rhythmic bending and unbending of the inner tube throughout the expiration phase); intrapulmonary percussive ventilation devices (also known as IPV devices that provide continuous oscillation to the airways via the mouth, endotracheal tube, or nose); and other devices that provide forced oscillation or impulse oscillometry. In one embodiment, an IPV device that applies vibratory air pressure waves superimposed on the breath airflow is used to generate and/or maintain oscillating airflow to a subject. Examples of such IPV devices are described in U.S. Pat. Nos. 6,595,213 and 6,695,978, both of which are incorporated herein in their entirety. In another embodiment, an oscillating device such as that disclosed in U.S. Pat. No. 4,333,476 is used to generate and/or maintain oscillating airflow to the subject.

The methods and devices described above for inducing intra-thoracic oscillations and generating and/or maintaining oscillating airflow to a subject are preferably applied as a superimposed oscillating pressure-flow force to normal breathing for single or multiple breaths. Such methods and devices can also be applied with large breaths and forced breaths. In certain embodiments, oscillation is applied to a subject only during inhalation or only during exhalation. In such embodiments, one configuration of the breathing circuit is to use a non-breathing valve to separate the inhalation and exhalation tubes. In other embodiments, oscillation is applied to a subject during both inhalation and exhalation. Following initial application, the frequency and amplitude of the pressure-flow oscillation applied to a subject is adjusted to optimize the concentration of EBC to be collected.

According to the invention, oscillating frequency can range from 0.5-1,000 hz. In one embodiment, the oscillating frequency is in the range of 5-100 hz. In another embodiment, the oscillating frequency is in the range of 10-300 hz. In certain embodiments, the oscillating amplitude is in the range of 1-15 cm H₂O pressure. In other embodiments, the oscillating volume ranges between 5-20% of total lung capacity. The amount of time oscillating forces are administered to a subject will be determined by the amount of sample to be collected. In one embodiment, the oscillating forces are administered to a subject from about 1 minute to 1 hour. In another embodiment, the oscillating forces are administered to a subject from about 5 minutes to 20 minutes. Those skilled in the art can readily adjust oscillating frequency, amplitude, volume, and amount of administration time in relation to the subject, lung capacity, and airway diameters.

In one embodiment of the subject invention, the frequency and amplitude of the oscillating pressure can be constant based on the optimum frequency for moving airborne molecules or lung/airway particles into the EBC. In a related embodiment, the frequency of oscillating pressure is varied to the optimum frequency for moving airborne molecules or lung/airway particles into the EBC. In this embodiment, the frequency and amplitude is adjusted to airway as a function of the trachea diameter and total lung capacity, which are important for the size of the subject (such as human versus other animal species and adult versus child sizes).

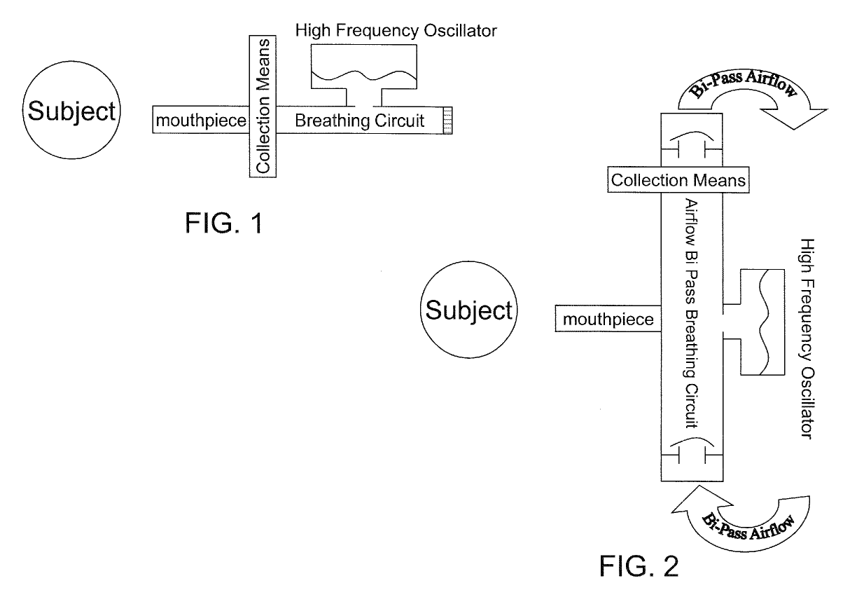

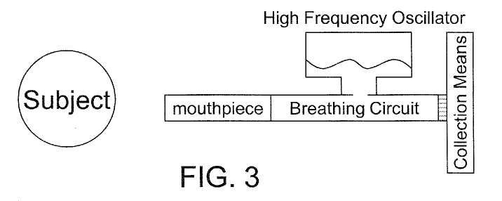

In a preferred embodiment, the external means for generating and/or maintaining oscillating airflow to the subject is a device that uses the same principle as a loud speaker or piston pump. One oscillating system for use in accordance with the invention includes the Jaeger Master Screen Impulse Oscillator System (Viasys, Inc.). This device uses a fixed frequency and amplitude oscillation of a speaker attached as a side arm to a breathing circuit. An oscillating electrical current is applied to a speaker (or piston pump) to generate an inflating and deflating pressure force. This force is applied as a side-arm on a breathing circuit (or tube) through which the subject is inhaling and/or exhaling (see FIGS. 1-3). As illustrated in FIGS. 1-3, the pressure force is superimposed on the airflow through the tube (or circuit) that is the breathing air produced by the subject.

According to the invention, external oscillating airflow is applied as a superimposed oscillating pressure-flow force to normal breathing for single or multiple breaths. The oscillator is applied with large breaths and/or forced breaths. As illustrated in FIGS. 1-3 the outflow of the subject's air passes over the collection means. The oscillation can be present during both inhalation and exhalation, although oscillation can also be applied only during inhalation or only during exhalation. The oscillation can also be applied during breathing behaviors such as cough, large breaths and forced exhalations.

Following application of oscillating pressure-flow force to a subject, the outflow (or exhaled breath) from the subject is directed to at least one collection means (as depicted in FIGS. 1-3). The collection means is any suitable containment method or device for containing a sample of EBCs taken from a subject's outflow (exhaled breath). For example, the collection means can be a receptacle for collecting volatile and/or non-volatile components of EBC. Such receptacles include, but are not limited to, tubes, vials, strips, capillary collection devices, cannulas, and miniaturized etched, ablated or molded flow paths. The collection means can be a material, such as an absorbent material, used to collect liquids. Examples of absorbent material for use in accordance with the invention include, but are not limited to, sponge-like materials, hydrophilic polymers, activated carbon, silica gel, activated alumina, molecular sieve carbon, molecular sieve zeolites, silicalite, AIPO₄ alumina, polystyrene, TENAX series, CARBOTRAP series, CARBOPACK series, CARBOXEN series, CARBOSEIVE series, PROAPAK series, SPHEROCARB series, DOW XUS series, and combinations thereof. In certain embodiments, the collection means can include both a receptacle and material described above. Those skilled in the art will know of other suitable receptacles and absorbent materials for use in accordance with the invention.

In certain embodiments, the collection means is any one of many known devices for extracting condensates from exhaled breath, generally involving a cooling process and/or gravitational forces and/or specific flow characteristics (such as narrowing a portion of the device to produce high turbulent flow rates and thus cooling of the sample) to condense the condensates for aqueous phase glucose analyses. For example, condensate can be collected from a subject's sample of exhaled breath using a device that relies on gravity to form a condensate pool or a device that exposes the sample of exhaled breath to cool temperatures. Gravity-based devices require that condensate droplets become large enough to overcome water's natural tendency to stick to the walls of a collecting tube. Eventually, the amount of condensate in the collection area becomes large enough for detection and/or analysis.

In those devices using a cooling process, the collection receptacle is sometimes inserted into an ice bucket or may even be separately cooled by refrigeration systems in order to increase the amount and speed of condensate formation. In an embodiment of the invention, a Peltier device is placed in contact with one wall of an EBC collection means and cooled so that EBC preferably condenses in the cooled area of the collecting device. In some cases, the collection means is a tube and a coating such as TEFLON™ is applied to make the tube walls non-wetting and non-reactive with EBCs and to enhance the speed and amount of condensate collected.

In certain embodiments, collected EBC samples are subjected to sensors for detection and/or quantification of biomarkers present in the sample. Sensors of the subject invention can include commercial devices commonly known as “artificial” or “electronic” noses or tongues. Other sensors for use in accordance with the subject invention include, but are not limited to, metal-insulator-metal ensemble (MIME) sensors, cross-reactive optical microsensor arrays, fluorescent polymer films, surface enhanced raman spectroscopy (SERS), diode lasers, selected ion flow tubes, metal oxide sensors (MOS), bulk acoustic wave (BAW) sensors, calorimetric tubes, infrared spectroscopy, semiconductive gas sensor technology; mass spectrometers, fluorescent spectrophotometers, conductive polymer gas sensor technology; aptamer sensor technology; amplifying fluorescent polymer (AFP) sensor technology; microcantilever technology; molecularly polymeric film technology; surface resonance arrays; microgravimetric sensors; thickness sheer mode sensors; surface acoustic wave gas sensor technology; radio frequency phase shift reagent-free and other similar micromechanical sensors.

Some fractions of an exhaled breath (also referred to herein as outflow from a subject) can yield different concentrations of certain EBC than other fractions. For example, the first one-third to one-half of an exhaled breath comprises mostly air that has been inhaled into the test subject's upper airway, but never gets into the deep lungs, where gas exchange takes place. Therefore, concentrations of EBCs that originate in the deep lungs are higher in later fractions of the exhaled breath than in earlier fractions. Therefore, for some types of EBCs targeted for detection in diagnosis according to the invention, it may be desirable to select only the later fractions of the exhaled breaths for collection and to divert earlier fractions away from the collection means or processes. This feature of the invention can be implemented in a number of ways, including, but not limited to, detection and use of markers, for example, carbon dioxide concentration, which is also higher in the later fractions of the inhaled breath, to control such flow diversions or to turn collection means and processes on and off. It can also be implemented by measuring volume fractions, for example, by measuring and diverting the first 30 percent, 50 percent, or greater, away from the collection means or process or turning on the collection means or process for the remaining exhaled breath fraction. Another implementation may be time, by timing breaths and activating collection after a selected time interval from the start of a breath.

It is also important to remove large-mass artifacts from the exhaled breath, such as food particles, sputum, expectorate, saliva, and the like, before collecting the exhaled breath EBCs, because such artifacts can also contaminate and skew EBC detection results, regardless of exhaled breath volume control. The air outflow collection procedures described herein can also provide for conditioning the exhaled breath by removal of such artifacts before exposing the exhaled breath to the collection means and processes.

The following example illustrates materials and procedures for making and practicing the invention. This example should not be construed as limiting. All percentages are by weight and all solvent mixture proportions are by volume unless otherwise noted. It will be apparent to those skilled in the art that the example involves use of materials and reagents that are commercially available from known sources, e.g., chemical supply houses, so no details are given respecting them.

### Example 1

Dogs with veterinary clinical diagnosis of lung disease and bacterial pneumonia will be anesthetized. Their exhaled air will be sampled with three protocols in this example: 1) the dogs will be anesthetized and mucosal surface nasal and oral samples will be collected directly by means of sterile probe, 2) the dogs will then quietly breathe through our collection filter for 10-20 minutes, 3) the high frequency air pressure oscillation (HFO) experimental protocol will then be presented as the dog breathes through a collection filter with the HFO device superimposing the air pressure oscillation on the normal tidal breath to vibrate the lung airway.

The dogs will be prepared for an anesthetized diagnostic procedure. An intravenous catheter will be placed and anesthesia induced with a slow intravenous bolus (over at lest 1 minute to prevent apnea) of propofol (4-8 mg/kg). This will be followed by an infusion of propofol (0.1-0.4 mg/kg/min), with the rate adjusted to maintain an appropriate level of anesthesia. The animals will be intubated. When a sufficient plane of anesthesia has been established, a sterile probe (swab) will be used to collect nasal and oral mucosal surface samples directly.

Following sampling of nasal and oral mucosal surface, the animals will have a non-rebreathing valve with an expiratory filter attached to the endotracheal tube. The dog will quietly breathe through the non-rebreathing valve, exhaling into a collection filter for 10-20 minutes.

Then, the HFO breathing device will be attached to the center chamber of the non-rebreathing valve and a new collection filter put into place. Air pressure will be oscillated at 10-300 hz. The animal will breathe spontaneously with the FIFO superimposed on the normal tidal volume. The animals will exhale through the collection filter with the HFO vibrating the lung airway for 5-20 minutes.

Following the experiment, all of the swabs and breathing filters collected in the experiment will be stripped for 10 minute in 10 ml phosphate buffer saline at room temperature, and the stripping solution will be then centrifuged at 8500 g for 10 minutes at room temperature. The supernatant will be decanted and stored for volatile organic compounds using HPLC and mass spectrometry. 100 ul phosphate buffer saline will be added to the precipitate pellets. The pellets will be analyzed via real-time PCR, microscopy, colony-forming assay and proteomic assays.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

I claim:

1. A method for increasing the concentration of exhaled breath condensates (EBC) in exhaled breath comprising: supplying an external means for generating and/or maintaining an oscillating airflow to a subject; collecting at least one exhaled breath sample from the subject following application of the oscillating airflow; and collecting an EBC sample from the exhaled breath sample.

2. The method of claim 1, further comprising the step of identifying and/or measuring the concentration of at least one biomarker present in the EBC sample.

3. The method of claim 2, further comprising the step of identifying any diseases or conditions associated with the biomarker(s) present in the EBC sample.

4. The method of claim 3, wherein the biomarker is one associated with lung cancer.

5. A system for increasing the concentration of EBC in exhaled breath comprising: an external oscillating means for application to a breathing circuit to a subject; and a collection means for collecting at least one exhaled breath and EBC sample from the subject.

6. The system of claim 5, further comprising a sensor for detecting any biomarker present in the EBC sample.

7. The system of claim 6, wherein the biomarker is one associated with lung cancer.
